(19) United States
(12) Patent Application Publication
(10) Pub. No.: US 2025/0084441 A1
(43) Pub. Date: Mar. 13, 2025

(54) METHOD FOR PRODUCING HUMAN INDUCED PLURIPOTENT STEM CELLS THROUGH HOMOLOGOUS RECOMBINATION AND INTEGRASE-MEDIATED RECOMBINATION

(71) Applicant: HEMACELL BIOTECHNOLOGY INC., Suzhou, Jiangsu (CN)

(72) Inventors: Fangfang ZHU, Suzhou, Jiangsu (CN); Yibing JI, Suzhou, Jiangsu (CN); Chen WU, Wuxi, Jiangsu (CN)

(21) Appl. No.: 18/723,041

(22) PCT Filed: Dec. 24, 2022
(86) PCT No.: PCT/CN2022/141732

§ 371 Date: Jun. 21, 2024

(30) Foreign Application Priority Data
Dec. 24, 2021 (WO) PCT/CN2021/141336

(51) Int. Cl. C12N 15/90; C12N 5/074; C12N 15/85

(52) U.S. Cl. CPC C12N 15/907; C12N 5/0696; C12N 15/85

(57) ABSTRACT

Provided is a method of producing human induced pluripotent stem cells from human somatic cells, including a step of homologous recombination and a step of integrase-mediated reaction.

### FIELD OF THE INVENTION

The present disclosure provides a method for generating human induced pluripotent stem cells (hiPSCs) from human somatic cells by homologous recombination combined with integrase-mediated reaction.

### BACKGROUND

The current methods for generation of human induced pluripotent stem cells (hiPSCs) from somatic cells mostly rely on retrovirus- or lentivirus-mediated delivery of transgenes coding for a set of transcription factors, for example, the Yamanaka factors (OCT3/4, SOX2, KLF4 and c-MYC; “OSKM”) (Takahashi and Yamanaka, 2006), or the Thomson factors (OCT3/4, SOX2, NANOG and LIN28) (Yu and Thomson, 2007).

The problem with lentivirus or retrovirus mediated delivery of transcription factors is that, the transduced viral vector and transgenes will be randomly and stably integrated into human genome, which has a risk of insertion of multiple viral copies into human genome. These viral insertions cause heavy genomic modifications, which may lead to unwanted activation or inactivation of host genes, potentially impairing normal cell function or increasing the risk of tumorigenicity. Although the transgenes are silenced during the iPSC generation process through de novo DNA methylation, they may be spontaneously reactivated during cell culture and differentiation. Moreover, different transcription factors will have different expression levels in different cells or even in the same cells, rendering the low efficiency of iPSC generation. These drawbacks of the lentivirus- and retrovirus-mediated random integration raise serious issues for both basic research and clinical applications.

Karow et al. conducted site-specific reprogramming in murine by inserting a reprogramming cassette into fibroblast cells and adipose stem cells by means of phiC31 integrase-mediated recombination at “pseudo attachment (att) site” in murine genome (M. Karow et al., Stem Cells. 2011 November; 29 (11): 1696-1704). The reprogramming genes in the resulting iPSCs were removed by Cre recombinase mediated excision to reduce tumorigenicity. The authors did not prove the feasibility of the method in human cells in this article. This method has several drawbacks which may not be suitable for application on human cells. Specifically, “pseudo att site” is a sequence that naturally exists in the recipient genome and shares certain sequence identity with the genuine att site of phiC31 integrase, so that it can be recognized by the integrase to accomplish the integrase-mediated recombination without an additional step to pre-integrate attachment site in the recipient genome. However, human genome is large and may have more than one sequence in the genome that can serve as pseudo att site, and some of them may locate at intragenic sites. Integration at intragenic site leads to loss of gene function. Multiple integration sites in the genome reduce the preciseness as compared to integration via true att sites. Also, integration at pseudo att sites may render deletion near the integration site or even chromosome rearrangement.

There remains a need for developing a method of generating iPSCs from human somatic cells with above-mentioned drawbacks being addressed.

### SUMMARY OF THE INVENTION

The present inventors developed a methodology for producing iPSCs from human somatic cells, comprising a step of enabling a homologous recombination between a donor plasmid and a safe harbor site in the genome of somatic cells to introduce a set of reprogramming genes as well as integrase attP sites at the safe harbor site, and a step of enabling integrase-mediated reaction to remove the reprogramming genes along with plasmid sequences from the iPSCs, or to replace them with gene(s) of interest after generation of iPSCs, thus completing the invention. The method of the present invention introduces the elements required for homologous recombination, reprogramming and integrase-mediated recombination in one step by one nucleic acid construct.

Accordingly, in a first aspect, the present disclosure provides a nucleic acid construct, comprising from 5′ to 3′:
i. 5′ homology arm, which is homologous to a first region of a locus,
ii. a first attachment site of integrase I,
iii. a reprogramming cassette, comprising coding sequences of a set of reprogramming factors,
iv. a first attachment site of integrase II, and
v. 3′ homology arm, which is homologous to a second region of the locus, wherein the locus is a safe harbor locus in human genome.

In a second aspect, the present disclosure provides a vector, e.g. a plasmid vector or a composition comprising the nucleic acid construct of the first aspect. The vector of the second aspect (donor vector of the homologous recombination) can be used to introduce the reprogramming cassette flanked by the first attachment sites of integrases into the safe harbor locus of the genome of human somatic cells.

In a third aspect, the present disclosure provides a second vector comprising a second attachment site of integrase I and a second attachment site of integrase II, and optionally a gene of interest cassette between the two attachment sites. The second vector (donor vector of the integrase-mediated recombination) can be used to remove the inserted reprogramming cassette or replace the inserted reprogramming cassette with a gene of interest via integrase-mediated reaction in the presence of corresponding integrases.

In a fourth aspect, the present disclosure provides a kit, comprising the vector of the second aspect, the second vector of the third aspect, and one or two integrase expression vectors comprising coding sequences of the integrases.

In a fifth aspect, the present disclosure provides a method of producing induced pluripotent stem cells (iPSCs) from human somatic cells, comprising:
a) introducing the vector of the first aspect into the human somatic cells,
b) culturing the human somatic cells under conditions that facilitate recombination between the 5′ homology arm and the first region and between the 3′ homology arm and the second region, resulting in insertion of the reprogramming cassette in the locus, and generation of master somatic cells,
c) culturing the master somatic cells generated by step b) under conditions that facilitate reprogramming to allow generation of master iPSCs, and
d) optionally introducing a second vector and an integrase expression vector into the master iPSCs generated by step c), wherein the second vector comprises a second attachment site of integrase I and a second attachment site of integrase II, and optionally a gene of interest cassette between the two attachment sites, and wherein the integrase expression vector comprises a coding sequence of the integrase I and a coding sequence of the integrase II; and
e) optionally culturing the iPSCs generated by step d) under conditions that facilitate integrase-mediated recombination between the first and second attachment sites of integrase I and between the first and second attachment sites of integrase II, resulting in excision of reprogramming cassette from the genome of iPSCs, and generation of iPSCs without the reprogramming cassette and optionally with a gene of interest.

In a sixth aspect, the present application provides a population of iPSCs generated by the method of the fifth aspect.

In a seventh aspect, the present application provides use of any of the vectors in the preparation of iPSCs.

The present invention has several advantages over currently available reprogramming system, specifically as follows:
The homologous recombination occurs at safe harbor site, significantly reducing the risk of gene disruption resulted from random integration of reprogramming genes.
The integration of reprogramming genes is precise, not only because the location of integration can be predetermined, but also because iPSCs as generated will have only a single copy of each integrated gene in the genome. Therefore, the reprogramming factors will be expressed at similar levels in all iPSCs.
The reprogramming genes and plasmid materials can be readily removed by transient expression of integrases after reprogramming.
The removed reprogramming genes can be replaced by gene(s) of interest during the integrase-mediated reaction, providing numerous potential applications.
The method can be used to reprogram a vast range of cell types, including blood cells such as PBMCs. Reprogramming blood cells is known to be a challenging task due to the heterogeneity of blood cell population and uneasy cell expansion.

### BRIEF DESCRIPTION OF THE DRAWINGS

FIG. 1 is a schematic showing the generation of iPSCs from somatic cells by introducing reprogramming factors via integrase-mediated recombination.

FIG. 2 shows the GFP expression that indicates the successful transfection of plasmid.

FIG. 3 shows the morphology of colonies.

FIG. 4 shows the immunocytochemistry staining of pluripotent markers of iPSC colonies.

### DETAILED DESCRIPTION OF THE INVENTION

### Definitions

Unless specifically defined elsewhere in this document, all the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

“iPSCs” as used herein refers to pluripotent stem cells (PSCs) generated by reprogramming of somatic cells. PSCs, including iPSCs and ESCs (embryonic stem cells) are characterized by their ability to self-renew and differentiate into any cell types.

“Somatic cell” refers to a cell of a living organism other than reproductive cells.

“Master somatic cells” or “Master iPSCs” as used herein respectively refer to somatic cells or iPSCs integrated with reprogramming cassette.

“PMBC” is the abbreviation of peripheral blood mononuclear cell.

“Cassette” or “expression cassette” as described herein refers to a DNA component included in a vector (e.g., a plasmid vector), and consisted of one or more genes (e.g., genes of reprogramming factors) to be expressed in a host cell transfected by the vector and regulatory sequence(s).

“Reprogramming” as described herein refers to the process of reverting somatic cells into induced pluripotent stem cells.

“Reprogramming cassette” as described herein refers to an expression cassette comprising a nucleotide sequence coding for one or more of reprogramming factors. The genes of the reprogramming factors are referred as “reprogramming genes”. “Linker sequence” exists between any two consecutive reprogramming genes. The reprogramming cassette can further comprise a reporter gene, a selection marker or the like.

“Donor plasmid” or “donor” refers to the plasmid carrying the sequences to be introduced into a genome via recombination. In the homologous recombination step of the present method, the donor plasmid comprises a nucleic acid comprising the reprogramming cassette. In the recombination mediated by integrase, the donor plasmid comprises a nucleic acid comprising the gene(s) of interest. In some cases, the donor plasmid in the context of the present application specifically refers to the donor plasmid for the homologous recombination which introduces the reprogramming cassette into human genome, while the donor plasmid for the integrase-mediated recombination is referred as “gene of interest (GOI) plasmid”.

“Target genome” refers to the genome intended to be modified, in particular by introducing an exogenous sequence via homologous recombination or integrase-mediated recombination.

“Safe harbor site” or “SHS” as used herein refers to an intergenic or intragenic locus which is transcriptionally active in the target genome. By “safe” it means that modification, in particular insertion, at such site is unlikely to cause disruption of normal gene functions or phenotypic changes of the host.

“Homologous recombination” refers to exchange of strands between two DNA molecules based on sequence homology. Homologous recombination naturally occurs in cells and plays an important role in repairing DNA double strand breaks.

“Integrase” refers to phage integrases that conduct recombination between attachment (att) sites in bacterial and phage genomes. The att site in bacterial genome is known as attB, while the att site in phage genome is known as attP. The integration usually requires phage integrase to work with bacterial host factors. Certain integrases such as phiC31 and Bxb1 function independently of the host factors. Accordingly, they can work in cellular environments different from its native bacterial environment, and have become powerful tools to conduct site-specific recombination in cells from bacteria to mammals.

“att site” refers to attachment site for the integrase of interest. When reference is made to integrase in the present application, att site is used interchangeable with the term “recognition site” or sometimes “recombination site”

“Gene of interest” or “GOI” refers to the gene under discussion. In the context of the present method, GOI specifically refers to the gene to be introduced into the genome of iPSCs via integrase-mediated reaction.

“Site-specific integration” means that the integration of an exogenous nucleic acid at a pre-determined location, instead of a random site, in the host genome. In the context of the present application, the homologous recombination and integrase-mediated recombination both lead to site-specific integration of incoming genes.

### Homologous Recombination

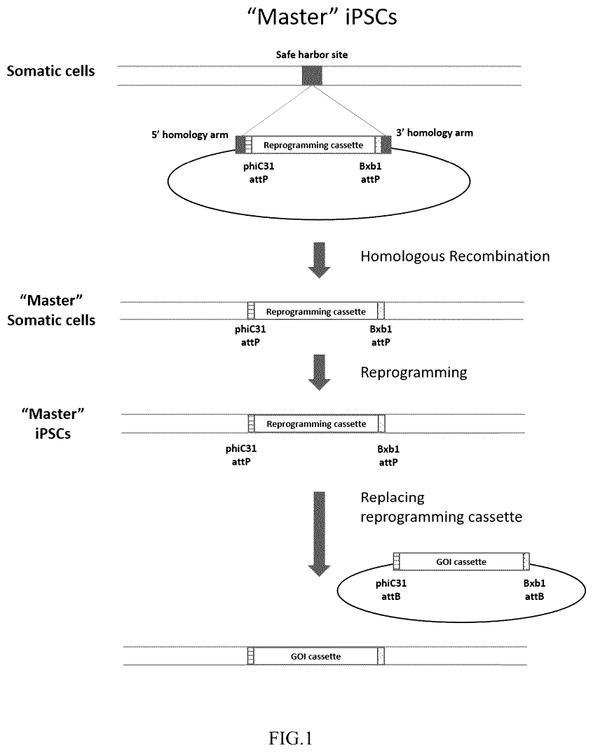

As shown in FIG. 1, the method of the present application comprises two steps of essential recombination reactions. The first recombination reaction is a homologous recombination between a region in the genomic sequence of a somatic cell and a donor vector carrying the reprogramming genes, by which the reprogramming genes are introduced into the genome without disrupting normal gene functions, resulting in generation of master somatic cells of the present application. After the first step of recombination, the master somatic cells can be reprogrammed into master iPSCs. Then the second step of recombination mediated via integrase occurs between the master iPSCs and a donor vector carrying an “empty” construct or a gene of interest (GOI) so as to remove the reprogramming genes or replace the reprogramming genes with GOI, resulting in iPSCs which do not comprise any exogenous programming genes, and may further comprise one or more genes of interest.

In the context of the present application, the homologous recombination can be achieved by any method known in the art or developed in the future as long as it can achieve the above mentioned results, i.e. introduction of the reprogramming genes at a desired location in the genome, e.g. at a safe harbor site.

In one embodiment, the homologous recombination can be a spontaneous homologous recombination. By “spontaneous homologous recombination” it means that the recombination spontaneously occurs in a cell without the aid of any artificially introduced exogenous enzyme. When the homologous recombination is spontaneous homologous recombination, the homology arms comprised in the donor plasmid are designed based on the sequence of the region comprising position of insertion.

Researches have revealed that DNA double-strand breaks (DSB) can enhance the efficacy of homologous recombination. Therefore, creating DSB at targeted site, by e.g. nuclease, would be desirable.

Accordingly, in some embodiments, the homologous recombination of the present application can be mediated by nucleases such as TALEN, ZFN, or Cas9.

For example, the homologous recombination can be TALEN-assisted recombination.

“TALEN” refers to Transcription activator-like (TAL) effector nuclease, which can be used to conduct gene editing by creating double-strand breaks. TALEN comprise a TAL effector DNA-binding domain and a DNA cleavage domain. The DNA-binding domain comprises 33-35 amino acids, which can be engineered to target and bind to a specific DNA sequence as desired, which will be cut by the DNA cleavage domain of TALEN.

When the homologous recombination is TALEN-assisted recombination, the homology arms comprised in the donor plasmid of the homologous recombination are designed based on the sequence of the region where DSB will occur in the safe harbor site. Also, in addition to the donor plasmid comprising the reprogramming genes, additional plasmids encoding TALEN elements are required to complete the homologous recombination of the present method. The TALEN elements include TAL proteins which recognize and bind to specific nucleotides, and FokI nuclease which makes the DSB.

For example, the homologous recombination can be ZFN-mediated recombination. By “ZFN” it refers to zinc finger nuclease, which comprises a zinc finger DNA-binding domain and a DNA-cleavage domain. Similar to TALEN, ZFN can be engineered to target specific DNA sequences as desired.

For example, the homologous recombination can be conducted by CRISPR/Cas system, e.g. CRISPR/Cas9 system. As one of the most popular gene editing tools, CRISPR/Cas9 comprises an editable single-guide RNA (sgRNA) sequence and an endonuclease Cas9 (CRISPR-associated protein 9). Site-specific editing is accomplished by engineering the sgRNA. The present application also contemplate the use of other CRISPR associated endonucleases discovered in recent years, such as Cas12a, Cas13a or the like.

The homologous recombination of the present application occurs in vitro, preferably under a condition that facilitate the occurrence of homologous recombination.

### Homologous Recombination-Donor Plasmid

The donor plasmid involved in the homologous recombination comprises a cassette of reprogramming genes.

The reprogramming cassette comprises nucleotide sequences encoding for a combination of transcription factors required for accomplishing the reprogramming. The combination of transcription factors can be any one known in the art or proven to be efficient in the future.

Non-limiting examples of reprogramming factors include POU5F1 (OCT3/4), NANOG, SOX2, LIN28A, KLF4, MYCL, MYCN, MYC, p53 knockdown, MIR302/367cluster, ESRRB, REX1, GBX2, DLX4, ZSCAN10, ZSCAN4, TBX3, GLIS1, NR5A1/2, RARG, BMI1, KDM2B, TET1SV40L, TGBX2, NANOGP8, SP8, PEG3 and ZIC1. The reprogramming cassette of the present application can comprise one or more genes encoding for one or more of aforementioned reprogramming factors. In preferred embodiment, the donor plasmid or the reprogramming cassette at least comprises the gene encoding OCT3/4. In another preferred embodiment, the donor plasmid or the reprogramming cassette at least comprises the genes encoding OCT3/4 and SOX2.

In specific embodiments, the combination of transcription factors can be selected from a group consisting of (1) Yamanaka factors, OCT3/4, SOX2, KLF4 and c-MYC (OSKM); (2) OCT3/4, SOX2, and KLF4 (OSK); (3) Thomson factors, OCT3/4, SOX2, NANOG and LIN28. The genes of the reprogramming factors can be arranged in any order in the cassette. A certain order may be preferable than others. For example, as for OSKM factors, starting with the OCT3/4 gene and ending with the c-MYC gene is preferred. However, it should be understood by one skilled in the art that the merit of the invention lies in the design of the reaction scheme instead of the choice and arrangement of reprogramming factors.

The nucleotide sequences encoding the reprogramming factors can be spaced by a nucleotide sequence coding for a linker. In one embodiment, the linker can be IRES (internal ribosome entry site), such as mini IRES, EMCV IRES (IRES from encephalomyocarditis virus) or mutant. In another embodiment, the linker can be a self-cleaving peptide, such as 2A peptide, including but not limited to T2A, F2A, E2A, P2A or the like. The advantage of IRES is that it does not produce any peptide fragment. However, the cistrons linked by IRES may not be expressed at a comparable level. It has been reported that the gene located upstream of IRES will be expressed at a significantly higher level than the one located downstream of IRES. Rather, viral 2A peptides, when used as a linker to connect two or more genes, can produce approximately equimolar levels of protein products. Accordingly, in a preferred embodiment, a nucleotide sequence of 2A peptide is used as the linker sequence to separate the reprogramming genes.

The genes of reprogramming factors can be transcribed polycistronically under the control of a single promoter. Exemplary promoters can include EF1α, CMV, ACTB, PGK, UbC or CAG. But one skilled in the art that any conventional promoter can be used in the reprogramming cassette.

In addition, the reprogramming cassette can comprise one or more of the following elements: replication origin, selection marker, and 3′ untranslated sequence. For example, replication origin can be F1 ori. Selection marker can be a gene conferring resistance to certain antibiotic, such as puromycin-resistant gene. 3′ untranslated sequence can be a poly-adenylation sequence (Poly(A)), e.g. TK poly A or SV40 poly A.

The reprogramming cassette can further comprise a reporter gene to indicate the successful insertion of the reprogramming cassette. The reporter gene can be one encoding the fluorescent protein, e.g. green fluorescent protein (GFP), yellow fluorescent protein (YFP), cyan fluorescent protein (CFP), or bioluminescence, e.g. luciferase, or the like.

The reprogramming cassette is flanked by two att sites, e.g. two attP sites or two attB sites, to facilitate recombination, as the att sites are recognition sites of the integrase. During recombination, each att site breaks in the middle, and two recombinant att sequences, attL and attR are each formed by an att fragment from the donor and an att fragment from the target genome. The att site can be a naturally occurring att site originally derived from phage, or a functional variant or fragment thereof which can be recognized by the integrase and can mediate the recombination reaction. In preferred embodiment, the att site is more than 16 bp in length to avoid it matches to any random sequence in the genome and reduce the specificity of the site. For example, the att site can be any length ranging from 17 bp to 60 bp (any integer within this range).

Donor plasmid of the homologous recombination further comprises two homology arms flanking the attP-reprogramming gene(s)-attP construct or attB-reprogramming gene(s)-attB construct. Each of the homology arms of the present application is homologous to a region of the genome so as to trigger homologous recombination.

The two homology arms can be homologous to two non-overlapping fragments of the safe harbor site, e.g. two non-overlapping fragments of H11 locus, respectively. When the locus of insertion is an intragenic position of a non-essential gene, two homology arms can be homologous to two non-overlapping fragments of the non-essential gene.

The length of homology arms may vary to a large extent from about 10 bp to 10 kb, or even longer, e.g. about 50 bp to 500 bp, about 100 bp to 400 bp, e.g. about 10 bp, 20 bp, 30 bp, 40 bp, 50 bp, 100 bp, 200 bp, 300 bp, 400 bp, 500 bp, 600 bp, 700 bp, 800 bp, 900 bp, or 1000 bp. Two homology arms may have similar length or different length.

A homology arm in the donor plasmid may not be 100% identical to its corresponding region in the genome to allow homologous recombination to take place. In some embodiment, the homology arm has at least 50% sequence identity, at least 60% sequence identity, at least 70% sequence identity, at least 80% sequence identity, at least 90% sequence identity to the corresponding genomic region, e.g. a fragment of safe harbor site or non-essential gene. In preferred embodiment, each of the homology arm has at least 90% sequence identity, such as at least 91%, at least 92%, at least 93%, at least 94%, at least 95%, at least 96%, at least 97%, at least 98%, at least 99%, at least 99.5%, to the corresponding genomic region, e.g. a fragment of safe harbor site or non-essential gene.

Depending on the mechanism of recombination, e.g. spontaneous or nuclease mediated, the selection of genomic regions to which the homology arms correspond from a certain safe harbor site or a non-essential gene, as well as the design of specific sequences of homology arms, can depend on different rules.

For example, when nuclease-mediated recombination is used, homology arms can be sequences which are suitable for the usage of the nuclease-mediated recombination system.

The two regions in the genome corresponding to the two homology arms, respectively, can be consecutive fragments or can be spaced by one or more nucleotides.

### Homologous Recombination-Locus of Insertion

In the present method, insertion of the reprogramming cassette into the genome does not impact the normal function of the cell. This can be realized by inserting the exogenous sequence into a locus in the genome known as “safe harbor”.

In some embodiments, the locus of insertion is an intergenic safe harbor site. A safe harbor site in human genome can be one selected from those known in the art, e.g. a safe harbor site recited in Stefan Pellenz et al. (“New Human Chromosomal Sites with ‘Safe Harbor’ Potential for Targeted Transgene Insertion”, HUMAN GENE THERAPY, VOLUME 30 NUMBER 7, 2019).

It has been suggested that efficiency of recombination varies with the choice of safe harbor site. The location of safe harbor sites may also influence the continuous expression of the transgene inserted therein. Certain safe harbor sites show a better performance in maintaining transgene expression as compared to others. In the present application, the safe harbor site is the location where the reprogramming factors inserts. A high recombination efficiency and a more stable expression of reprogramming factor are both desirable for the present application. In a preferred embodiment, the safe harbor site in human genome of the present application can maintain a continuous expression of the transgene. In a preferred embodiment, the homologous recombination at the safe harbor sites can result in recombination of a high efficiency.

The safe harbor site especially useful in the present method includes but not limited to H11 (Hipp11), AAVS1, hROSA26, and CCR5. More preferably, the safe harbor site in human genome is H11 site.

In some embodiments, the safe harbor site can be an intragenic region of a non-essential gene. By “non-essential gene” it means the normal function of the cell of interest would not be significantly impacted when the gene loses its function. For example, the locus of insertion can be within such genes as TRAC, TRBC1, TRBC2 or the like.

After the locus of insertion is determined, homology arms used to mediate the homologous recombination can be designed based on the sequence of the region around the locus.

### Homologous Recombination-Introduction of Donor Plasmid

The method of the present application comprises a step of inserting the reprogramming cassette of the present application into the genome of somatic cells via homologous recombination.

Introduction of donor plasmid can be conducted by any conventional means in the art. For example, the donor plasmid comprising the reprogramming genes can be transfected into the somatic cells by various methods including biological methods, chemical methods and physical methods. Exemplary biological transfection is transfection mediated by viruses such as adeno virus, adeno-associated virus (AAV), herpes simplex virus (HSV) and the like. Examples of chemical transfection are calcium phosphate transfection, calcium chloride transfection, and transfection using cationic polymer or cationic lipids. Examples of physical transfection include direct injection, biolistic particle bombing, and electroporation. One skilled in the art can determine the method of transfection.

### Homologous Recombination-Culture Condition

Homologous recombination can be spontaneous homologous recombination or nuclease-assisted homologous recombination occurred in cultured cells in vitro. The suitable cell culture conditions are known to one skilled in the art.

For the homologous recombination of the present application, there's no special restriction on the culture condition of the cells, as long as the culture condition can support the growth of the cells. One reason is that the efficiency of homologous recombination is not crucial in the whole methodology, although a higher recombination efficiency is usually favorable. Cells will be subjected to identification and selection at a later stage. Only those cells with reprogramming factors successfully inserted can be reprogrammed into pluripotent stem cells.

Suitable culture condition can be determined based on the type of cells. Any somatic cells can be used as the starting point of the present method, into which the reprogramming plasmid is introduced. For example, the somatic cells can be fibroblast cells which are commonly used for reprogramming. In another embodiment, the starting somatic cells are blood cells which are not conventionally used for reprogramming. The blood cells suitable for the present method can be isolated from bone marrow, umbilical cord blood or peripheral blood, e.g. peripheral blood mononuclear cells (PBMCs) or CD34⁺ hematopoietic stem and progenitor cells.

Exemplary medium for culturing blood cells include but not limited to Iscove's Modified Dulbecco's Medium (IMDM) supplemented with fetal bovine serum (FBS) or human plasma, or RPMI 1640 Medium supplemented with fetal bovine serum (FBS) or human plasma, AIM V Serum Free Medium (Gibco), MarrowMAX™ Bone Marrow Medium (Gibco), Blood Cell Growth Medium (Sigma-Aldrich), or StemSpan™ Serum-Free Expansion Medium (STEMCELL Technologies). One or more growth factors and cytokines which support the growth of blood cells can be supplemented in the culture medium. As to CD34⁺ cells isolated from human PBMCs, the isolated cells can be cultured in a medium, e.g. StemSpan™ Serum-Free Expansion Medium II (STEMCELL Technologies), or StemPro™-34 SFM (Gibco), supplemented with one or more cytokines or growth factors, preferably all of hSCF, hTPO, hIL-3, hIL-6 and hFlt3L.

After the introduction of the donor plasmid, the cells can be maintained under a condition suitable for somatic cells for a period of time, e.g. 2-10 days, 3-8 days, or 4-6 days, before switching to a condition suitable for inducing reprogramming.

### Homologous Recombination-Verification

To select the master somatic cells, which are cells with introduced donor plasmid, the cells can be cultured under selection stress when the donor plasmid comprises a selection marker.

Successful recombination can be verified by conventional means in the art. For example, successful introduction of the construct of homology arm-attP-reprogramming gene(s)-attP-homology arm or homology arm-attB-reprogramming gene(s)-attB-homology arm into a somatic cell can be confirmed by PCR, sequencing, and/or southern blot. In another embodiment, for donor plasmid or reprogramming cassette comprising a reporter gene, successful introduction of the construct can be verified based on the presence of reporter gene product.

Alternatively, no verification is needed after the introduction of donor plasmid and before the reprogramming step is completed. Verification assays can be postponed to a later stage after the somatic cells have been cultured under reprogramming conditions for a suitable period of time.

### Reprogramming

After the introduction of reprogramming cassette, the master somatic cells can be cultured under a condition to induce reprogramming so as to produce master iPSCs. Reprogramming condition can include use of culture medium which helps to induce the cell reprogramming. Such medium may comprise certain compounds and/or nutrients which facilitate the reprogramming or enhance the efficiency of reprogramming. Such compounds includes but not limited to cytokines, growth factors, minerals, etc., Said nutrients include but not limited to essential and non-essential amino acids.

Exemplary medium suitable for reprogramming process can include DMEM/F12 as the basal medium, and further include supplements such as serum supplements, amino acid supplements, minerals and so on. The supplements can be commercially available premix e.g. N-2 Supplement, B-27™ Supplement, GlutaMAX, or can be formulated on site. Specific examples of medium can be DMEM/F12 supplemented with 10% KnockOut Serum Replacement (KSR), MEM Non-essential Amino Acids Solution, GlutaMAX, β-Mercaptoethanol, and 100 ng/ml basic FGF; or DMEM/F12 supplemented with 1% N-2 Supplement, 2% B-27™ Supplement, MEM Non-essential Amino Acids Solution, GlutaMAX, β-Mercaptoethanol, and 100 ng/ml basic FGF; or mTeSR™Plus Medium (Stemcell).

The cells can be switched to a reprogramming culture condition with or without verification of successful introduction of the reprogramming cassette, and cultured under reprogramming condition for a suitable period of time, e.g. at least 48 hours. In some embodiments, the cells are cultured in reprogramming medium for 14-21 days.

The cells will be cultured in conditions suitable for somatic cells within 48 hours of introduction of donor plasmid. After that, the same amount of reprogramming medium (the same as iPSC culture medium) will be added to the somatic cells, making a 1:1 ratio of the somatic cell culture medium and the reprogramming medium. 48 hour after that, complete medium will be removed from the somatic cells, and then only reprogramming medium will be used for all the future culture.

### Reprogramming—Verification

iPSC clones can only be generated after successful homologous recombination. iPSC clones can be cultured in the same medium as used for reprogramming or a different medium suitable for iPSCs. Specific medium for iPSCs can be DMEM/F12 supplemented with 10% KnockOut Serum Replacement (KSR), MEM Non-essential Amino Acids Solution, GlutaMAX, β-Mercaptoethanol, and 100 ng/mL basic FGF; or DMEM/F12 supplemented with 1% N-2 Supplement, 2% B-27™ Supplement, MEM Non-essential Amino Acids Solution, GlutaMAX, β-Mercaptoethanol, and 100 ng/ml basic FGF; or Essential 8™ Medium (Gibco). iPSC clones with successfully introduced reprogramming cassette are referred as “master iPSCs” and can be verified by conventional means in the art, including but not limited to PCR and sequencing, or by southern blot.

For verification by PCR and sequencing method, gDNA can be extracted from iPSC clones. For example, multiple pairs of primers can be designed, specifically as follows: Pair 1: forward primer: a sequence of 20 bp at 300 bp outside of the 5′ arm of the homology arm, reverse primer: a sequence in the reprogramming cassette OCT3/4 gene; Pair 2: forward primer: a sequence in the reprogramming cassette c-MYC gene, reverse primer: a sequence of 20 bp at 300 bp outside of the 3′ arm of the homology arm; Pair 3: forward primer: a sequence of 20 bp at 300 bp outside of the 5′ arm of the homology arm, reverse primer: a sequence of 20 bp at 300 bp outside of the 3′ arm of the homology arm. PCR products are subjected to electrophoresis. Correct and single allele targeted clones would produce a band of about 600 bp in PCR reactions with primer pair 3, and a band of about 2 kb in PCR reactions with primer pairs 1 and 2. Further confirmation can be conducted by sequencing to verify that no unwanted mutations exist in the selected clone.

Additionally, Southern blot can be conducted to confirm the identity of inserts in the iPSC clones.

To verify the pluripotency of generated iPSCs, any conventional means known in the art can be used. For example, the identity of iPSCs can be verified by immunocytochemistry staining for pluripotency markers and/or teratoma assay.

### Integrase-Mediated Reaction—Integrases

As shown in FIG. 1, the second recombination reaction comprised in the present method is an integrase-mediated reaction occurring between a donor plasmid (GOI plasmid or empty plasmid) comprising integrase attachment sites, e.g. attB sites and the genomic region already inserted with the reprogramming genes flanked by two corresponding integrase attachment sites, e.g. attP sites. The recombination relies on the existence of corresponding attachment sites in the donor plasmid and the genomic region and the presence of integrase(s). Two attachment sites are needed either on the donor plasmid or in the genomic region, as the sequence between the two attachment sites will be exchanged by the recombination. One skilled in the art would understand that the type of attachment sites in the donor plasmid of the integrase-mediated reaction and the type of attachment sites flanking the reprogramming genes can be switched, as long as one is attB and the other is attP.

In one embodiment, the two attachment sites can be the same attachment sites for a single integrase to recognize. In this case, only one integrase is needed to complete the recombination. However, the drawback of one integrase-mediated recombination is that the sequence from the donor plasmid can be inserted into the genome in either direction, cutting the chance of obtaining correct recombination product into half theoretically.

Accordingly, in a preferred embodiment, the integrase-mediated recombination involves two integrases and the flanking attachment sites at either side of the sequence to be exchanged are different. As a result, the direction of the inserted sequence can be controlled.

The integrase of the present application is not naturally expressed in human cells. The integrase can be delivered and expressed by a plasmid, which is referred as “integrase plasmid” in the present application. For the two integrases system, two plasmids can be used in the method, each encoding for one of the integrases. In a preferred embodiment, the integrase is transiently expressed in the host cell, specifically master iPSCs.

Exemplary integrases can be serine recombinases (serine integarses), including but not limited to phiC31 integrase derived from bacteriophage phiC31, Bxb1 integrase derived from mycobacteriophage, TP901-01 recombinase derived from phage TP901-01, spoIVCA recombinase from Bacillus licheniformis, or R4 integrase derived from phage R4 of Streptomyces parvulus.

In specific embodiments, the integrase of the present application can be selected from Bxb1 integrase and phiC31 integrase.

For example, for one integrase system, the sequences to be exchanged are flanked by the same attachment sites in the same plasmid. For example, either Bxb1 integrase or phiC31 integrase can be used in one integrase system. Take Bxb1 integrase as an example for illustration. In the reprogramming plasmid, two Bxb1 attp sites flank the reprogramming genes, while in the GOI plasmid, two Bxb1 attb sites flank the GOI or any sequence to be inserted into the genome to replace the reprogramming cassette. Only one integrase plasmid is required to deliver the coding sequence of Bxb1 integrase into the host cell.

Another example is two-integrase system, which is more preferred in the present application. In the two-integrase system, Bxb1 integrase and phiC31 integrase are both involved in the integrase-mediated recombination. In the reprogramming plasmid, Bxb1 attP site locates at one side (e.g. 5′ upstream) of the reprogramming genes and phiC31 attP site locates at the other side (e.g. 3′ downstream) of the reprogramming genes. In the GOI plasmid, Bxb1 attB site locates at one side (e.g. 5′ upstream) of the gene of interest (if any) and phiC31 attB site locates at the other side (e.g. 3′ downstream) of the gene of interest (if any). For the integrase-mediated recombination, coding sequences of both Bxb1 and phiC31 are delivered into the cell by either one or two plasmids.

### Integrase-Mediated Reaction—GOI Plasmid and Integrase Plasmid

More than one plasmid are involved in the integrase-mediated reaction, including a donor plasmid comprising the attachment sites e.g. attB sites and one or two integrase plasmid carrying the gene encoding for integrase(s).

The donor plasmid of the integrase-mediated reactions comprises two attachment sites e.g. attB sites corresponding to the attachment sites e.g. attP sites comprised in the donor plasmid of the homologous recombination reaction.

To simply remove the reprogramming genes from the genome of iPSCs, no sequence needs to exist between the two attachment sites, e.g. attB sites. In one embodiment, the sequence can be the original sequence existed between the two regions to which the two homology arms correspond in the genome. Then the integrase-mediated reaction will restore the original sequence at the locus of insertion.

To replace the reprogramming genes with gene of interest (GOI), the sequence between the two attachment sites, e.g. attB sites in the donor plasmid of this step is the sequence of GOI. In the present application, GOI can be any gene of interest. For example, it can be a disease related gene, a mutant gene, a reporter gene, a development related gene, and so on.

### Integrase-Mediated Reaction—Reaction Condition

Master iPSCs can be dissociated into single cells before being subjected to further process. The dissociation can be completed by a physical method or a chemical method e.g. by using enzyme such as accutase. Also, the master iPSCs can be cultured under conditions as described previously in the section entitled “Reprogramming”, supplemented with chemicals that increase the survival of iPSC as single cells, e.g. ROCK inhibitors Y-27632, Thiazovivin, or the like.

Plasmids carrying GOI and integrases can be introduced via conventional means of transfection in the art as described above.

### Integrase-Mediated Reaction—Verification

The integrase-mediated reaction aims to remove the reprogramming genes from the genome of iPSCs. Therefore, verification of the completion of this step can be conducted by observation of loss of reporter gene, or by PCR reaction of the targeted regions.

Uses of iPSCs

The iPSCs as generated by the present method have multiple uses in scientific research and drug development.

For example, in case that the integrase-mediated recombination removes the reprogramming genes from the iPSCs, the resulted iPSCs free of any exogenous genes can be used on any occasions where iPSCs are needed.

In some embodiment, the integrase-mediated recombination replaces the reprogramming genes with gene of interest, which expands the application of the iPSCs to infinite possibilities. For example, the iPSCs with the introduced GOI can be used to study gene functions, gene mutations and interactions in differentiation and more. For example, the introduced GOI may be a disease-related gene so that the generated iPSCs can be used for treating a disease, e.g. by gene therapy. Potential applications also include drug screening, in vitro disease modeling, cell therapy and so on.

### Examples

In following part, exemplary experimental protocols are provided.

In one exemplary embodiment, the present application can be carried out as follows and reference can be made to FIG. 1.

Following constructs were prepared by molecular cloning:
a) a donor plasmid, comprising 5′ H11 homology arm-phiC31 attP-genes of reprogramming factors-GFP gene-Bxb1 attP-3′ H11 homology arm
b) a GOI plasmid, comprising phiC31 attB-genes of interest-Bxb1 attB
c) integrase expression plasmid, comprising coding sequence of phiC31 integrase and coding sequence of Bxb1 integrase.

Human somatic cells were cultured in appropriate medium before use.

The donor plasmid was delivered into human somatic cells with electroporator or nucleofector, and mark this as Day 1. The transfected somatic cells were plated onto either mouse embryonic fibroblast (MEF) feeder cells or onto Matrigel/fibronectin-coated cell culture plate, and cultured in appropriate medium for 2 days. At day 3, the same volume of iPSC culture medium was added to the cells for 2 days.

At day 5, the medium was switched to human iPSC culture medium. Medium was changed every day with fresh human iPSC culture medium and the cell culture was continued until desired morphology of human iPSC colonies can be observed.

Individual human iPSC colonies were picked. Each individual colony was cultured and expanded separately. The picked human iPSCs were characterized with one or more methods selected from immunocytochemistry for pluripotency markers, DNA methylation analysis for OCT3/4 promoter, in vitro differentiation into three germ layers, teratoma assay, southern blot to integration copy number assay, and DNA sequencing to make sure reprogramming factors from the donor plasmid were inserted into H11 locus, etc, and several candidate clones were identified.

The phiC31 and Bxb1 integrase expression plasmid and gene of interest plasmid were delivered into the candidate human iPSCs to allow integrase-mediated recombination between the attP sites in H11 locus and attB sites in gene of interest expression plasmid with the help of phiC31 and Bxb1 integrases, and the reprogramming factors from the donor plasmid will be deleted from the H11 locus while the genes of interest would be inserted into the H11 locus for later use.

Similar assays as described above were conducted to identify the final clones.

### Verification of Successful Introduction of Reprogramming Cassette

iPSC clones with successful homologous recombination were verified by (1) PCR and sequencing and (2) southern blot:

### 1. PCR and Sequencing

Extract gDNA from the iPSC Clones

Do PCR with the following three pairs of primers using the extracted gDNA as the template: Pair 1: forward primer: a sequence of 20 bp at 300 bp outside of the 5′ arm of the homology arm, reverse primer: a sequence in the reprogramming cassette OCT3/4 gene; Pair 2: forward primer: a sequence in the reprogramming cassette c-MYC gene, reverse primer: a sequence of 20 bp at 300 bp outside of the 3′ arm of the homology arm; Pair 3: forward primer: a sequence of 20 bp at 300 bp outside of the 5′ arm of the homology arm, reverse primer: a sequence of 20 bp at 300 bp outside of the 3′ arm of the homology arm.

Run the PCR product on the agarose gel. Correct and single allele targeted clones would produce a band of about 600 bp in PCR reactions with primer pair 3, and a band of about 2 kb in PCR reactions with primer pairs 1 and 2. Select these clones for sequencing in the fourth step.

Purify the bands of 2 kb from PCR reactions with primer pairs 1 and 2 from clones selected in step 3, and do sequencing to identify clones that have no mutations.

The clones with correctly introduced reprogramming cassette were used for southern blot analysis.

### 2. Southern Blot

Extract gDNA from the iPSC Clones

Digest the DNA into fragments with restriction enzymes, and separate the DNA fragments on agarose gel.

Transfer the DNA fragments from the gel onto a nylon membrane, and wash the nylon membrane with a prehybridization solution containing salmon sperm DNA to block non-specific DNA interactions and reduce background noise.

Prepare the probe targeting the promoter region in the construct and label with 32p alpha-labeled dCTP.

Incubate the blot with labeled probe, and detect the probe and the DNA sequence of interest by exposure to film.

Only iPSC clones with one band in southern blot were correctly targeted clones and used for further characterization.

### Reprogramming

Reprogramming was conducted according to the following protocol.

Isolate CD34+ cells from human PBMC.

Plate CD34+ cells onto 24 well plate and culture in StemSpan™ Serum-Free Expansion Medium II supplemented with hSCF, hTPO, hIL-3, hIL-6 and hFlt3L.

Three days later, transfect cell with the reprogramming plasmid using Etta™ X-Porator H1 system or the Lonza™ 4D-Nucleofector™. Plate transfected cells onto Corning™ matrix-coated culture dishes, and incubate them in StemSpan™ medium containing cytokines.

Two day after transfection, add same volume of mTeSR™Plus iPSC culture medium to the transfected cells, and culture for another 2 days.

Four days after transfection, change the medium to mTeSR™Plus Medium and monitor the culture vessels for the emergence of iPSC colonies. Replace the spent medium every other day.

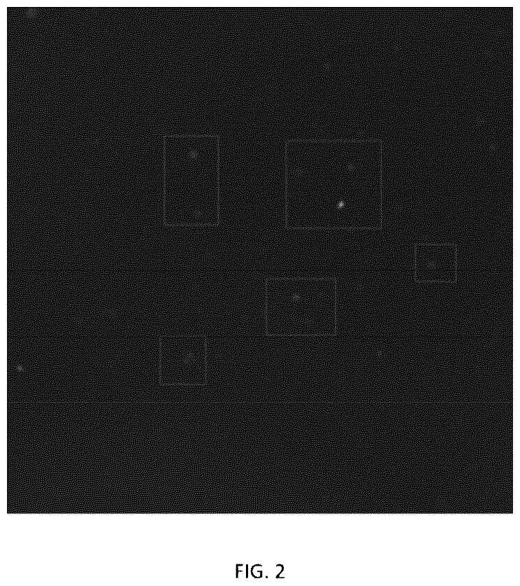

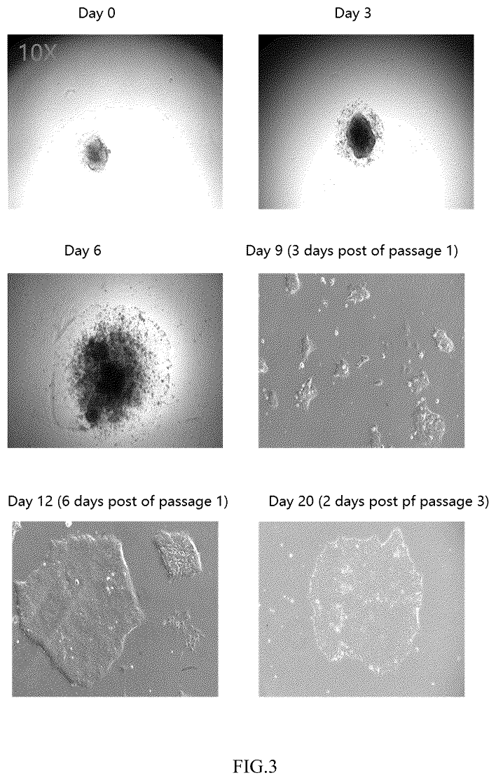

The plasmid construct carried neomycin resistance and GFP genes for selection and screening. After transfection, GFP expression provided an indication of the level of gene expression from the locus as shown in FIG. 2. Two to three weeks after transfection, GFP+ undifferentiated iPSCs were picked and transferred onto fresh Corning™ matrix-coated culture dishes for expansion and passage. After culture and passage for 3 times (around 20 days), these colonies displayed typical hES cell morphology as shown in FIG. 3.

### Immunocytochemistry Staining for Pluripotency Markers

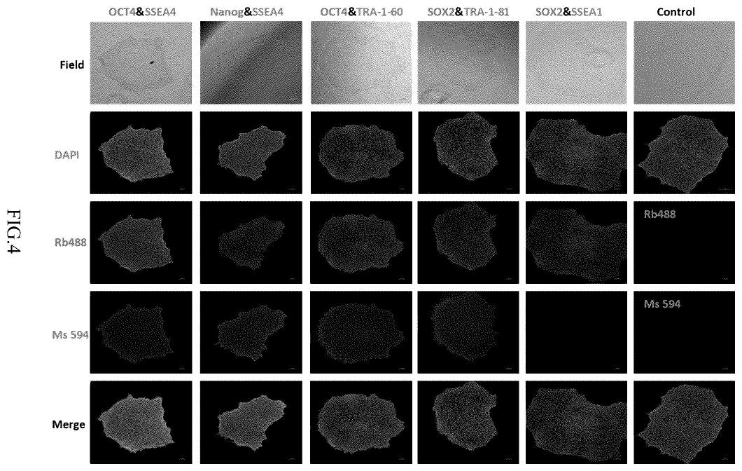

The cells were tested for pluripotency markers by immunocytochemistry according to the following protocol. These cells expressed the pluripotent cell surface markers TRA-1-60, TRA-1-81 and SSEA-4, in addition to OCT4, SOX2 and NANOG (FIG. 4).

Briefly wash cells once with PBS, and fix cells with 4% paraformaldehyde in PBS for 15 minutes.

Rinse cell with PBS, and permeabilize and block using 2.5% donkey serum in PBST (PBS+0.1% TritonX-100) for 60 minutes at room temperature.

Incubate cells with the following primary antibodies diluted in 2.5% donkey serum in PBST at 4° C. for 16-18 hours: SSEA4, TRA-1-60, TRA-1-81, OCT4, SOX2, NANOG.

After washing cells three times with PBS, incubate cells with the following secondary antibodies diluted in PBS containing 0.1% bovine serum albumin at room temperature for 1 hour: Rhodamine-labeled donkey anti-mouse IgG, Rhodamine-labeled donkey anti-rabbit IgG, and Rhodamine-labeled donkey anti-goat IgG.

Wash cells three time and stain for the nuclei with 1 μg/ml DAPI.

After three washes, observe the staining results under a fluorescence microscope.

Harvest iPSCs by 1 mg/ml ReLeSR™ treatment, and centrifuge and resuspend cells in DMEM/F12 medium.

Subcutaneously inject cells from one 60 mm dish into a non-obsess diabetes/sever-combined immunodeficient (NOD/SCID) mouse.

Teratoma will form after six to eight weeks.

After two more weeks, sacrifice mice and dissect tumors.

Fix tumors with PBS containing 4% paraformaldehyde and embed with paraffin.

After slicing, stain the sections with hematoxylin and eosin.

Seed 5×10⁵ cells on 10 cm cell culture dishes for attachment. Cells were incubated at 37° C., 5% CO₂ for 48 h.

After 2 days of culture, add 0.1 ml colcemid, which can collapse mitotic spindles and prevent the completion of mitosis, to each dish and mix gently. Incubated at 37° C., 5% CO₂ for 2 h.

Transfer medium to centrifuge tubes from the cell culture dishes. Use PBS to wash the dishes, and remove PBS. Then, add 1 ml 0.1% Trypsine into the dishes at 37° C., 5% CO₂ for 2 min. Also, transfer the mixture (Trypsine and cells) into the centrifuge tubes and mix with the medium which is transferred to centrifuge tubes before we use PBS to wash the dishes. Further, centrifuge at 100 ref for 10 min at room temperature.

Discard the supernatant and leave 0.5 ml medium to mix the pellet gently.

Resuspend the pellet in 5-7 ml 37° C. hypotonic solution and mix thoroughly. Incubate in water bath at 37° C. for 10 min.

Centrifuge at 100 ref for 10 min at room temperature. Discard the supernatant and leave 0.5 ml solution to mix the pellet gently. Resuspend the pellet in 5 ml cold fixative (drop by drop and flick the tube between drops to prevent cell clumping).

Put the centrifuge tube on ice at least 20 min. Centrifuge at 100 ref for 10 min at room temperature. Discard the supernatant and add 3-5 ml cold fixative (Methanol and glacial acetic acid (3:1) to be made fresh and chilled before using).

Repeat the washing step three times or until the supernatant is and the pellet become white.

After the final centrifugation, suspend the cells in a few drops of cold fixative to give a slightly opaque suspension. Drop 1-2 drops onto wet and clean slides (Before that, the slides have to be rinsed by 1-2 drops of cold fixative).

Dry the slides at room temperature. Observe the chromosomes with the microscope (Olympus DP71 with 200× magnification). Count the number of chromosomes for about 50 cells. Chromosomal G-banding analysis of CD34+ PBMC-derived iPSCs showed a normal karyotype with no evidence of clonal abnormalities.

### Integrase-Mediated Recombination

### The Integrase-Mediated Recombination was Conducted According to the Following Protocol.

Dissociate iPSCs into single cells with accutase, and collect 1×10⁶ cells for the integrase-mediated reaction.

Transfect cells with 4 ug each of phiC31 integrase, Bxb1 integrase, and attB donor plasmid using the using Etta™ X-Porator H1 system.

Plate transfected cells onto Corning™ matrix-coated culture dishes and culture cells in mTeSR™Plus Medium supplemented with 2 uM Thiazovivin.

Change medium every day.

One week after transfection, pick and transfer GFP-undifferentiated iPSCs onto fresh Corning™ matrix-coated culture dishes for expansion. iPSCs with the reprogramming genes removed and further with the gene of interest introduced were successfully generated.

What is claimed is:

1. A nucleic acid construct, comprising from 5′ to 3′:
i. 5′ homology arm, which is homologous to a first region of a locus in human genome,
ii. a first attachment site of integrase I,
iii. a reprogramming cassette, comprising coding sequences of a set of reprogramming factors,
iv. a first attachment site of integrase II, and
v. 3′ homology arm, which is homologous to a second region of the locus,
wherein the locus is a safe harbor locus in human genome.

2. The nucleic acid construct of claim 1, wherein the integrase I and integrase II are integrases that are active in human cells.

3. The nucleic acid construct of claim 1, wherein the integrase I and integrase II are the same integrase or two different integrases.

4. The nucleic acid construct of claim 1, wherein the integrase I and the integrase II are selected from phiC31 integrase and Bxb1 integrase.

5. The nucleic acid construct of claim 1, wherein the first attachment site is attP or attB, preferably attP.

6. The nucleic acid construct of claim 1, wherein the safe harbor locus is H11 locus.

7. The nucleic acid construct of claim 1, wherein the 5′ and 3′ homology arms have a length of about 10 bp to 10 kb.

8. The nucleic acid construct of claim 1, wherein the reprogramming cassette further comprises a selection marker and/or a reporter gene.

9. The nucleic acid construct of claim 1, wherein the reprogramming cassette comprises the coding sequences of OCT3/4, SOX2, KLF4, and c-MYC.

10. The nucleic acid construct of claim 1, wherein the reprogramming cassette comprises from 5′ to 3′ the coding sequences of OCT3/4, SOX2, KLF4, and c-MYC.

11. The nucleic acid construct of claim 1, wherein the coding sequences of the reprograming factors are linked by a DNA sequence encoding a self-cleaving peptide.

12. (canceled)

13. A vector, comprising the nucleic acid construct of claim 1.

14. A composition, comprising the vector of claim 13.

15. A kit, comprising the vector of claim 13.

16. The kit of claim 15, further comprising a second vector comprising a second attachment site of integrase I and a second attachment site of integrase II.

17. The kit of claim 16, wherein the second vector comprises a gene of interest (GOI) cassette located between the second attachment site of integrase I and the second attachment site of integrase II, and said GOI cassette comprises a coding sequence of the gene of interest, wherein the GOI is selected from one or more of a group consisting of a therapeutic gene, a selection marker and a reporter gene.

18. (canceled)

19. (canceled)

20. The kit of claim 15, wherein the second attachment site is attB site when the first attachment site is attP, and wherein the second attachment site is attP site when the first attachment site is attB; preferably the second attachment site is attB.

21. (canceled)

22. A method of producing induced pluripotent stem cells (iPSCs) from human somatic cells, comprising:
a) introducing the vector of claim 13 into the human somatic cells,
b) culturing the human somatic cells under conditions that facilitate recombination between the 5′ homology arm and the first region and between the 3′ homology arm and the second region, resulting in insertion of the reprogramming cassette in the locus and generation of master somatic cells, and
c) culturing the master human somatic cells generated by step b) under conditions that facilitate reprogramming to allow generation of master iPSCs.

23. The method of claim 22, further comprising:
d) introducing a second vector and an integrase expression vector into the master iPSCs generated by step c), wherein the second vector comprises a second attachment site of integrase I and a second attachment site of integrase II, and the integrase expression vector comprises a coding sequence of the integrase I and a coding sequence of the integrase II; and
e) culturing the iPSCs generated by step d) under conditions that facilitate integrase-mediated recombination between the first and second attachment sites of integrase I and between the first and second attachment sites of integrase II, resulting in excision of reprogramming cassette from the genome of iPSCs.

24. (canceled)

25. (canceled)

26. (canceled)

27. (canceled)

28. (canceled)

29. (canceled)

30. (canceled)

31. (canceled)

32. (canceled)

33. (canceled)

34. A host cell comprising the nucleic acid construct of claim 1.

35. (canceled)
